(19) United States
(12) Patent Application Publication
(10) Pub. No.: US 2024/0398539 A1
(43) Pub. Date: Dec. 5, 2024

(54) ESOPHAGEAL STENTS AND RELATED METHODS

(71) Applicant: Merit Medical Systems, Inc., South Jordan, UT (US)

(72) Inventors: Zeke Eller, Plano, TX (US); Tiffany Ethridge, Lubbock, TX (US); Bryan K. Elwood, Arlington, TX (US)

(21) Appl. No.: 18/798,048

(22) Filed: Aug. 8, 2024

Related U.S. Application Data
(63) Continuation of application No. 17/383,221, filed on Jul. 22, 2021, now Pat. No. 12,090,038.
(60) Provisional application No. 63/056,471, filed on Jul. 24, 2020.

(51) Int. Cl. A61F 2/04; A61F 2/00

(52) U.S. Cl. CPC A61F 2/04; A61F 2002/0081; A61F 2002/044; A61F 2230/0069; A61F 2250/0039

(57) ABSTRACT

Stents are disclosed herein. The stents described herein can comprise a hollow cylindrical body in which a middle region extends to a first end and also extends to an opposing second end. The first end and the second end can each comprise a plurality of flanges, within which the flanges can have either similar or different characteristics, particularly physical profiles.

### RELATED APPLICATIONS

This application is a continuation of U.S. patent application Ser. No. 17/383,221, filed on Jul. 22, 2021 and titled ‘ESOPHAGEAL STENTS AND RELATED METHODS’, which claims priority to U.S. Provisional Application No. 63/056,471, filed Jul. 24, 2020, and titled ESOPHAGEAL STENTS AND RELATED METHODS, which are both incorporated herein by reference in their entireties.

### TECHNICAL FIELD

This application generally relates to medical devices. More particularly, this application relates to esophageal stents and related methods.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A shows a side view of an exemplary embodiment of a stent in an unelongated and unstretched state.

FIG. 1B shows a side view illustrating an alternative aspect of the exemplary embodiment shown in FIG. 1A.

FIG. 2A shows a longitudinal cross-section of a portion of the embodiment shown in FIG. 1A.

FIG. 2B shows a longitudinal cross-section of a portion of the embodiment shown in FIG. 1B.

FIG. 3 shows a perspective view of another embodiment of a stent.

FIG. 4A shows a side view of another exemplary embodiment of a stent in an unelongated and unstretched state.

FIG. 4B shows a side view illustrating an alternative aspect of the exemplary embodiment shown in FIG. 4A.

FIG. 5A shows a longitudinal cross-section of a portion of the embodiment shown in FIG. 4A.

FIG. 5B shows a longitudinal cross-section of a portion of the embodiment shown in FIG. 4B.

FIG. 6 illustrates an aspect of the embodiment shown in FIG. 5A.

FIG. 7 illustrates a side view of another embodiment of a stent.

FIG. 8 illustrates a side view of another embodiment of a stent.

### DETAILED DESCRIPTION

Stents are disclosed herein. In some embodiments, the stents described herein comprise a hollow cylindrical body having an interior dimension and comprising a middle region that extends to a first end and also extends to an opposing second end. The interior dimension refers to the three-dimensional space within the stent. As used herein, the hollow cylindrical body may refer to generally cylindrical shapes and forms, including stents with flared ends, for example. The first end and the second end can each comprise a plurality of flanges, within which the flanges can have either similar or different characteristics, particularly physical profiles.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As applied a stent deployed within an esophagus, the proximal end of the stent refers to the end closest to the patient's head, and the distal end of the stent refers to the opposite end, the end closer to the patient's stomach.

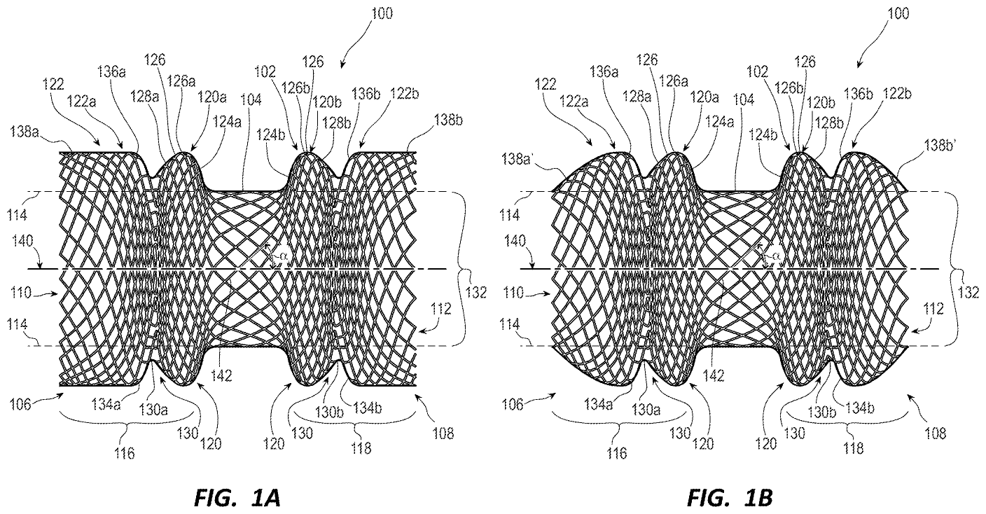

FIGS. 1A and 1B illustrate exemplary embodiments of a stent in an unelongated and unstretched state. The stent 100 comprises a hollow cylindrical body 102. The hollow cylindrical body 102 comprises a middle region 104 that extends to a first end 106 and also extends to an opposing second end 108. Two openings provide access to the interior of the hollow cylindrical body 102: a first opening 110 defined by the first end 106 and a second opening 112 defined by the second end 108.

In an aspect, one end of the stent body will be the proximal end and the other will be the distal end as defined herein. In some embodiments, the stent is configured so that either the first end or the second end can be proximal with the remaining end being distal based on the practitioner's choice. In other embodiments, use of the stent indicates that a specific end is consistently designated as the proximal end. In such embodiments described herein, the first end is intended for designation as the proximal end and the second end is intended for designation as the distal end unless expressly indicated otherwise.

In the illustrated embodiments, each of the ends comprises a plurality of flanges, flares, or flared regions that extend radially from the circumference 114 of the middle region 104. However, it should be noted that embodiments in which only one end includes these and other features described herein are also encompassed by the present disclosure. These flanges are configured to interact with the surfaces of a lumen in the body of a subject into which the stent 100 is deployed (e.g., such as the esophagus). More specifically, the first end 106 comprises a first plurality of flanges 116 and the second end 108 comprises a second plurality of flanges 118. As shown, each of the first plurality and the second plurality include at least two flanges. More specifically, the first plurality of flanges 116 includes an inner flange 120a adjacent to the middle region 104 and an outer flange 122a adjacent to the first opening 110, and the second plurality of flanges 118 similarly includes an inner flange 120b adjacent to the opposing end of the middle region 104 and an outer flange 122b adjacent to the second opening 112. As used herein in discussing the shapes and arrangement of flanges, “inner” refers to the side facing or in the direction of the middle region, while “outer” refers to the side facing or in the direction of the nearest opening. Additionally, while the embodiments shown herein depict a plurality of flanges on each end, it will also be appreciated that in some embodiments only one end of the stent includes a plurality of flanges, while the opposing end includes a singular flange.

FIGS. 1A and 1B also illustrate shapes for the flanges in accordance with various embodiments. In this and in subsequent figures, multiple occurrences of an element are indicated with a series of suffixes “a”, “b”, etc., added to a base reference number. Discussion herein of such an element that references its base reference number (e.g. 120) is understood to apply similarly to all occurrences (e.g. 120a, 120b) unless otherwise indicated. Likewise, relationships between two or more such elements (e.g., 120, 122) are understood to apply between occurrences indicated by the same suffix (e.g. 120a, 122a).

As shown in FIGS. 1A and 1B, the inner flange in each plurality can have a profile that is roughly triangular so as to form a hump-like structure protruding from the circumference of the middle region. In the embodiments shown, the profile of each of the inner flange 120 comprises an inner shoulder 124 extending radially from the circumference 114 to a peak 126 and an outer taper 128 returning from the peak 126 back toward the longitudinal axis 140 to a valley 130. In some embodiments, the diameter of the inner flange 120 at the peak 126 is at least about 2 mm greater than the diameter of the middle region 104. In more particular embodiments, the diameter of the inner flange 120 at the peak 126 is from about 2 mm greater to about 15 mm greater than the diameter of the middle region 104, or more particularly from about 3 mm to about 12 mm. In some embodiments, the diameter of the inner flange 120 at the peak 126 is between about 1.2 times and about 2.5 times the diameter of the middle region 110. In a particular aspect, the valley 130 defines an inner diameter that is at least equal to the diameter 132 of the middle region 104. In some embodiments, the inner diameter of the valley 130 can be somewhat or slightly larger than that of the middle region 104. For example, the diameter of the valley 130 can be between about 1.0 times and about 1.5 times larger than the diameter of the middle region 104.

As noted above, the outer flange 122 defines the adjacent opening into the interior of the stent. The profile of the outer flange 122 comprises an inner shoulder 134 extending from the circumference 114 or valley 130 to a peak 136. In some embodiments, the peak 136 of the outer flange 122 is the same height relative to the circumference 114 as the peak 126 of the inner flange 120. In certain embodiments, as exemplified by FIG. 1A, the outer flange 122 further comprises a cylindrical outer section 138 that is both concentric to the longitudinal axis 140 of the hollow cylindrical body 102 and parallel to the circumference 114, and that extends to the adjacent opening. In other embodiments as exemplified in FIG. 1B, the profile of the outer flange 122 is similar to that of the inner flange 120 in that the outer section 138 tapers toward the longitudinal axis 140 to define the adjacent opening.

In some embodiments, the overall length of the stent may range from about 30 mm to about 250 mm, including ranging from about 30 mm to about 70 mm or from about 160 mm to about 250 mm. In some embodiments, the length of the first end and/or the second end (which can include the plurality of flanges) in the unelongated and unstretched state may each range from about 10 mm to about 30 mm, including ranging from about 14 mm to about 22 mm. In some embodiments, the length of the middle region may range from about 5 mm to about 210 mm, including ranging from about 10 mm to about 30 mm and from about 30 mm to about 120 mm and from about 120 mm to about 210 mm. Smaller and/or larger stents are also contemplated depending on the desired use. For example, in some embodiments, the stents are configured for deployment within the esophagus. However, the disclosure is not so limited and other types of stents are also contemplated, including, but not limited to, gastrointestinal stents (including colonic stents), biliary stents, lung stents, vascular stents, transluminal stents, pancreatic stents, etc.

As shown in FIG. 1, the hollow cylindrical body 102 may comprise braided or woven wire 142. More specifically, the wires can be arranged in a particular braid pattern having a pitch, and with a braid angle a that can be constant over a given region of the body and also vary over other regions to provide certain shape and strength characteristics. In some embodiments, the braid pattern of the middle region 104 (and of the entire stent 100) is a one-wire, two-over, two-under braid pattern (referred to as a “one over two” pattern), which means that a single strand passes over two strands (or two different portions of itself, such as in a single wire braid design) and then under two other strands (or yet two other portions of itself, such as in a single wire braid design). Alternative braid patterns may be used as well, such as a one-wire, one-over, one-under braid pattern (referred to as a “one over one” pattern). Other possible braid patterns include the diamond two-wire, one-over, one-under braid pattern and the diamond two, two-over, two-under braid pattern.

In some embodiments, the braid pattern may lead to differing cell requirements over the length of the stent 100, where a cell refers to the design created by the braid pattern. Thus, depending on stent length and braid pattern, the braid designs may result in fractional and non-fractional cell counts. For example, a stent may be designed with a non-fractional cell count, in which a full braid pattern is completed on each end of the stent and/or the stent comprises only full braid patterns along the length of the stent. Non-fractional cell counts refer to a whole cell count. For example, a stent with a non-fractional cell count may have, 20, 30, 40, 50, or more full cell counts, or full braid patterns along its length. Fractional cell counts refer to fractional cell count numbers, 20.5, 30.5, 40.5, 50.5 or more, meaning the stent has a whole number of full cell counts in addition to a partial cell (or braid pattern) along the length of the stent. In some embodiments, the braid pattern may be one over one and may have a fractional or non-fractional cell count. In some embodiments, the braid pattern may be one over two and may have a fractional or non-fractional cell count.

As shown in FIGS. 1A and 1B, the braid angle a is an angle formed by a given strand of the braided or woven wire 142 relative to the longitudinal axis 140 of the stent 100. A larger (higher) braid angle, approaching, for example, 90 degrees, results in a higher pic count (number of points of intersection of the strands) per given longitudinal length (e.g., an inch) of a given braid (or weave) pattern. These parameters can be varied to impart certain characteristics to the stent body. A higher pic count can produce greater stiffness (i.e., a lower degree of compressibility). A smaller (lower) braid angle results in a lower pic count per given longitudinal length, which can result in greater softness (i.e., less stiffness and a higher degree of compressibility). In some embodiments, the braid angle α is from about 35° to about 75°, or more particularly from about 40° to about 70°. In certain of such embodiments, the braid angle α in the middle region is between about 35° and about 60°, and the braid angle α at the peaks of the flanges is between about 60° and about 70°. In some embodiments, the braid angle α is substantially constant over the middle region. In some embodiments, the braid angle α is substantially uniform at a peak of a flange while varying uniformly over the inner shoulder and the outer taper.

The pitch (i.e., axial distance between crossing strands) also impacts the compressibility and stiffness of the braided or woven wires. In an aspect, a sufficiently tight pitch can impart better migration resistance to the stent. The pitch is related to the number of strands interwoven (or interbraided) with each other and the braid angle a, and therefore can vary over different geometries. In some embodiments, the pitch is substantially constant over the middle region. In some embodiments, the pitch is substantially uniform at a peak of a flange while varying uniformly over the inner shoulder and the outer taper.

The braided or woven wires may be braided or woven in a given pattern in accordance with an appropriate braid design, such as a closed-loop braid design, a single wire woven design, an endless braid design, or the like. In some embodiments, the wire is braided in a closed-loop braid design in which multiple strands are interlaced in a first direction (e.g., a distal direction) and then turn and are interlaced back in an opposite second direction (e.g., back in the proximal direction). In still other embodiments, the stent may have an endless braid design in which multiple strands are interlaced. In some embodiments, the braid pattern can comprise hook stitches. In other embodiments, a “hook and cross” braid pattern is used in which the pattern includes both hook stitches and cross stitches. In some embodiments, the braid pattern is created using an axial braiding approach. In some embodiments, the braid pattern is created using a radial braiding approach. Radial braiding involves creating a fractional cell count along the length of the stent, either a non-fractional or a fractional cell count can be selected with axial braiding.

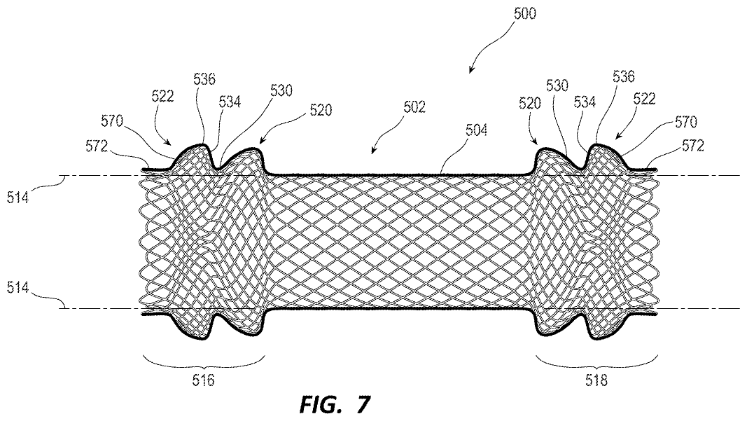

In various embodiments, the braided or woven wires are braided or woven so as to create a series of loops at one or both ends of the stent. The arrangement of these loops can be selected to provide desired functionality, such as for threading through of a suture line (discussed further below). In some embodiments the end loops are substantially aligned in one plane at the stent opening (see e.g., FIGS. 1A and 1B). In other embodiments, some end loops may protrude further than others. In certain embodiments, the position of the end loops can alternate (see e.g. FIG. 7).

The braided or woven wires may include varying numbers of strands, where the number used can depend in part upon the size of the stent and the braid or weave pattern. In some embodiments, the stent includes a wire count of from 32 to 64 wires, or more particularly from 45 to 50 wires, or more particularly from 48-56 wires.

When braided or woven in a closed-loop braid design, the braided or woven wires may start and stop at various locations on the stent 100. In an aspect, it can be advantageous to design the braid so that such termination points occur on surfaces that include a substantially flat dimension. In some embodiments, at least one wire termination point is on the middle region of the stent. In certain embodiments, such as the embodiment illustrated in FIG. 1A, at least one wire termination point is on the cylindrical outer section of an outer flange.

In various embodiments, the braided or woven wires forming the stent may comprise any suitable material known in the art, including plastics and memory alloys. In some embodiments, the wires may be Nitinol, including ASTM F2063. In certain embodiments, the thickness of a memory alloy strand of the braided or woven wires may be about 140 μm to about 191 μm. In other embodiments, the thickness may be about 165 μm to about 178 μm. Generally speaking, smaller wires may be used with smaller diameter stents and larger diameter wires may be used with larger diameter stents. Also, while smaller diameter wires can be used for stents designed for “through-the-scope” placement, stents designed for “over the wire” placement can include wires having significantly larger diameters. Radiopaque markers and/or coils can also be incorporated into the stent as desired.

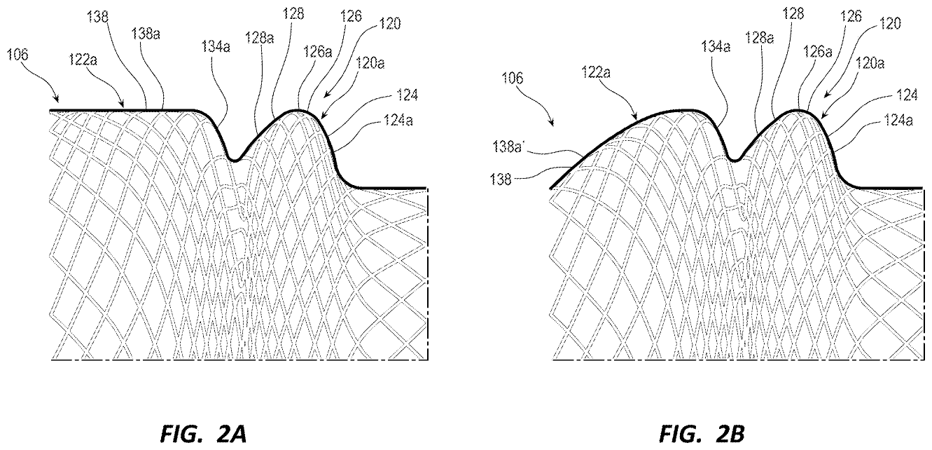

Additional aspects of the flanges in the embodiments described above are illustrated in FIGS. 2A and 2B, each of which show a cross-section of a portion of the first end 106 of one of the embodiments shown in FIGS. 1A and 1B, said portion defined by bisecting the hollow cylindrical body 102 both vertically and horizontally along the longitudinal axis 140. In some embodiments, an aspect of the inner shoulders of the various flanges is that they extend from the circumference 114 at an angle, slope, or arc that can be substantially perpendicular. An aspect of the inner flange 120 in particular is that the inner shoulder 124 includes a region that is substantially perpendicular to the longitudinal axis 140 while the outer taper 128 does not include such a perpendicular region. Another way to characterize the profile of the inner flange 120 is that the profile is asymmetric and includes a compound curve in which the different sections of the flange surface feature curves having different orientations and radii. In the illustrated embodiments, for example, the inner shoulder 124 includes a curve that is oriented more perpendicularly than a curve included in the outer taper 128, while the peak 126 includes a substantially horizontal curve having a smaller radius than the other curves. The profile of the inner flange 120 can also be described as an arc of an ellipse that is tilted inward from perpendicular, resulting in a hump having different slopes on its inner and outer faces. In yet further embodiments, the inner shoulder 124 can be described as extending outward at a steeper or more inclined angle, slope, or arc as compared to the outer taper 128 that extends inwards at a less steep or inclined angle, slope, or arc relative to the longitudinal axis of the stent.

The perpendicularly oriented region (or more perpendicularly oriented region having a steeper or more inclined angle, slope, or arc) of the inner shoulder 124 results in the peak 126 and, more generally the inner flange 120, exerting a hoop force against the tissue walls of a lumen in a subject so as to facilitate greater retention of the stent 100 in the body lumen. In another sense, the shape of the inner flange 120 functions as a shoulder region that resists longitudinal migration of the stent 100 due to peristalsis or other forces, in a manner that is independent of the magnitude of the hoop force. The shallower incline, angle, slope, or arc of the outer taper 128 increases stiffness of the inner flange 120 as compared to a flange that includes a perpendicular region on both inner and outer faces. For example, a flange having a symmetrical profile can tend to deflect more outwardly when in use, as compared to embodiments where the inner shoulders include more perpendicular regions, but the outer tapers do not include corresponding perpendicular regions.

The same aspects apply to each outer flange in embodiments in which said outer flange has a similar profile to that of the inner flange, such as with the outer flange 122a having a tapered outer section 138a′ in the embodiment illustrated in FIG. 2B. Similar effects are also provided in other embodiments in which the outer flange comprises a cylindrical outer section, such as the embodiment illustrated in FIG. 2A. That is, the inner shoulder 134 acts to facilitate greater retention of the stent 100 by exerting a force on the lumen walls, which is further facilitated by the increased stiffness contributed by the outer section 138. The cylindrical outer section 138 can provide the additional function of forming a seal with the lumen wall, such that material moving within the lumen passes into the opening defined by the outer section and through the body of the stent, rather than bypassing the opening and potentially getting stuck between the stent body and the lumen wall. The particular design of the outer flange can result in enhanced outward force exerted on the outer section, thereby increasing the quality of the seal.

In an aspect, the magnitude and nature of these effects can vary based on physical dimensions of the outer section, such as its length and diameter. For example, attaining such performance from a stent of a particular size may involve said stent including an outer section having a minimum length. On the other hand, increasing the length of the outer section means a concomitant increase in the overall length of the stent, which in turn can be limited by the particular clinical use. In various embodiments, the length of the outer segment can be selected so as to provide sufficient retention and sealing performance while keeping the overall stent length within applicable parameters for the intended use. Another consideration is provided where the cylindrical outer section is used as a termination surface for the woven or braided wire from which the stent body is made. Accordingly, in some embodiments, the outer segment has a length equal to at least one cell of the braid pattern.

The benefits of increased stiffness and strength provided by the flange shapes described above can be enhanced by combining two or more flanges in a “stacked” configuration in accordance with the various embodiments described herein. Without being bound or limited by a particular theory, the retention forces generated by the individual flanges can increase as a function of the number of flanges arranged together. Stated another way, in a plurality of flanges, one flange can increase the resistance of an adjacent flange to deformation and vice versa, thereby increasing the capacity of both flanges to resist migration of the stent.

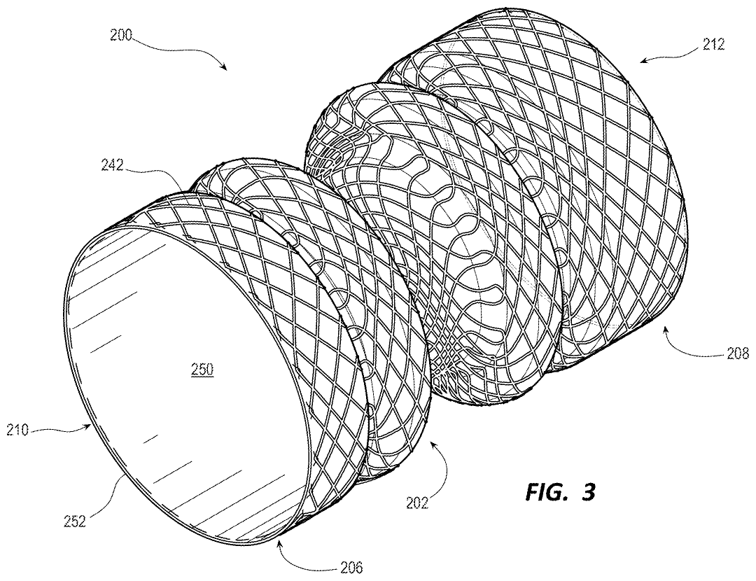

FIG. 3 shows another embodiment of a stent 200 including the features of the stent 100 shown in FIG. 1A, and further including a cover 250. The cover is coupled to the braided or woven wire 242 that forms the hollow cylindrical body 202. The cover 250 can further define the interior of the stent 200 and can facilitate passage of particles or fluid through the interior.

The cover may be elastomeric, polymeric, or comprised of any other material known in the art. In some embodiments, the cover may include silicone, while in certain embodiments the cover may be comprised only of silicone. In some embodiments, the cover may be applied such that it tends to ebb and flow into spaces between portions of the braided or woven wires, resulting in a “tire tread” like outer surface, rather than a smooth outer cover. This can allow tissue to lock into the uneven spaces and treads, thus adding anti-migration properties in some embodiments. In some embodiments, the cover is shaped such that the braided or woven wires are uncovered in some regions of the stent, which can allow tissue to grow into these spaces to help secure the stent in place. For instance, the peaks of the flanges can be at least partially uncovered, which can allow tissue ingrowth. In some embodiments, the cover can comprise two or more layers of material.

The stent 200 in FIG. 3 further includes a suture line 252 situated at the first end 206 of the hollow cylindrical body 202 and woven through the mesh created by the wire 242. As shown, the suture line 252 circumscribes the first opening 210. In some embodiments, the suture line 252 can be woven through end loops formed at an end of the hollow cylindrical body 202. The suture line 252 can be configured to aid with loading of the stent 200 into a catheter or other delivery device and can also facilitate removal of the stent 200 from a patient. The suture line 252 may be woven in such a way that pulling it has a drawstring effect on the first opening 210. That is, pulling a portion of the suture line 252 away from the first opening 210 causes the suture to draw the edges of the cover 250 and the first opening 210 radially inward, elongating and narrowing the hollow cylindrical body 202. In the case where the stent 200 is situated in a body lumen, this action facilitates removal by causing the stent 200 to disengage from the lumen wall. In some embodiments, a suture line can also be included at the second end 208 and can function similarly with respect to the second opening 212.

As discussed above, certain benefits can be realized in stents having two or more flanges in a stacked arrangement at each end, including an increased capacity to retain the stent's position in a lumen. Accordingly, in some embodiments a plurality of flanges can include one or more additional flanges situated between the inner flange and outer flange. For instance, one, two, three, four or more additional flanges may be disposed between the inner and outer flanges, resulting in a total of three, four, five, six, or more total flanges on an end of the stent. Further, in some embodiments, a proximal end of the stent can be configured with additional flanges (or more flanges) than the distal end to aid in preventing migration of the stent. Such an arrangement can be advantageous, although not required, when employing the stent in an esophagus.

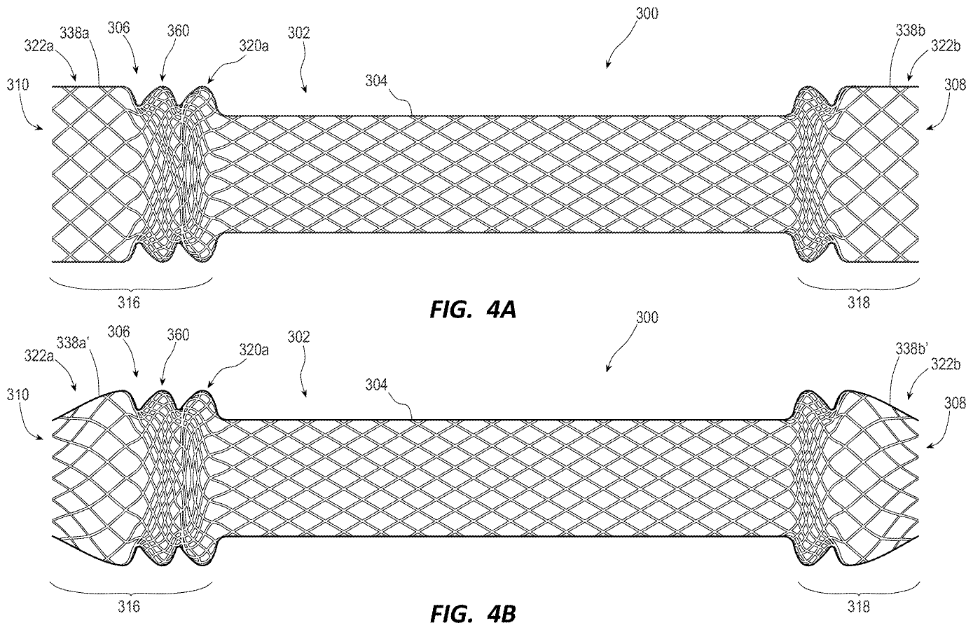

FIGS. 4A and 4B show an example embodiment of a stent 300 in which a hollow cylindrical body 302 comprises a middle region 304 that extends to a first end 306 and also extends to an opposing second end 308, and where the first end 306 comprises a first plurality of flanges 316 and the second end 308 comprises a second plurality of flanges 318. As shown, the first plurality of flanges 316 comprises an inner flange 320a adjacent to the middle region 304 and an outer flange 322a adjacent to the first opening 310, where the outer flanges 322a and 322b can feature an outer section 338a and 338b, respectively, that is cylindrical as shown in FIG. 4A, or a tapered outer section 338a′ and 338b′ respectively as shown in FIG. 4B. The first plurality of flanges 316 of this embodiment further comprises an additional flange 360 situated between these two. In some embodiments, as illustrated here, the additional flange 360 has a profile that is substantially the same as the inner flange 320a.

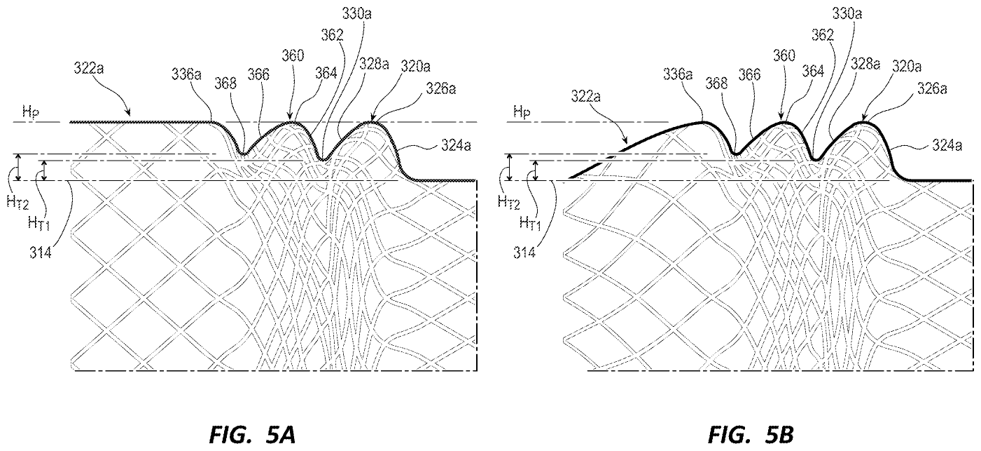

FIGS. 5A and 5B each show a cross-section of a portion of the first end 306 of the embodiments shown in FIGS. 4A and 4B, said portion defined using the same approach as in FIGS. 2A and 2B. As shown, a plurality of flanges, particularly one comprising three or more flanges, can include selected relative spatial and dimensional characteristics. The first plurality of flanges 316 of the embodiment shown in FIGS. 4A and 4B are shown in closer detail in FIGS. 5A and 5B, respectively. As shown, the profile of inner flange 320a comprises an inner shoulder 324a extending radially from the circumference 314 to a peak 326a and an outer taper 328a returning from the peak 326a back toward the longitudinal axis (not visible in this view) to a valley 330a. In the embodiment, the additional flange 360 has a similar profile, comprising an inner shoulder 362 extending radially from the valley 330a to a second peak 364, and an outer taper 366 returning from the peak 364 back toward the longitudinal axis to a second valley 368. In some embodiments, all of the peaks of a plurality of flanges are substantially the same height above the circumference of the middle region. In other embodiments, the peaks in a plurality of flanges can vary in height. In some embodiments, the height of the peaks varies in a random fashion. In other embodiments, the height of the peaks varies according to a pattern, such as alternating in height. This is illustrated in FIGS. 5A and 5B, where the peak 326a of the inner flange 320a, the peak 364 of the additional flange 360, and the peak 336a of the outer flange 322a all have substantially the same height Hp. In various embodiments, one or more valleys in a plurality of flanges sit at a level at least equal in height to the circumference of the middle region. Stated another way, the valleys define a part of the interior having an inner diameter substantially equal to that of the middle region. In some embodiments, the valleys can be situated above the level of the circumference of the middle region, i.e. define a diameter larger than that of the middle region. In some embodiments, the valleys can be situated below the level of the circumference of the middle region, i.e. define a diameter smaller than that of the middle region. FIGS. 5A and 5B illustrate a more particular embodiment in which the valleys become successively more shallow the more outwardly situated they are. Stated another way, each valley defines a diameter that is greater than any valleys closer to the middle region. As shown, valley 330a is situated at a height H_{T1} above the circumference 314 and defines a particular inner diameter, while valley 368 is situated at height H_{T2} above the circumference 314 and therefore defines a greater inner diameter.

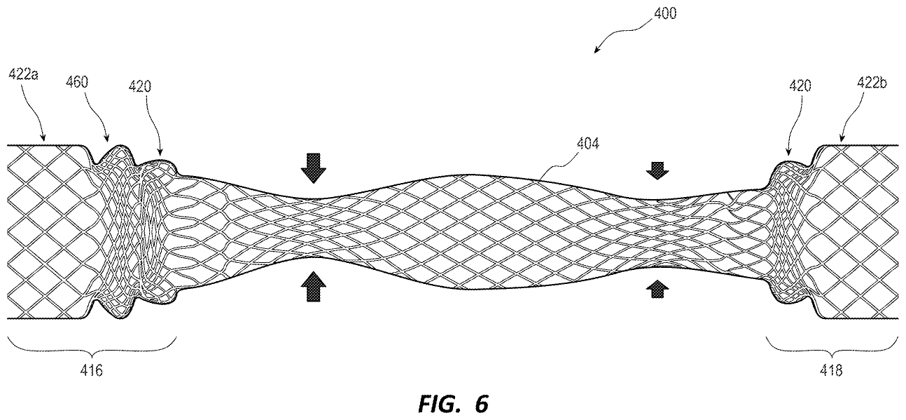

FIG. 6 illustrates the function of stents described herein. Specifically, FIG. 6 shows a stent 400 wherein the middle region 404 is undergoing compression, such as may occur in a body lumen due to peristalsis, tumor growth, or other constrictive phenomena. Without being bound by or limited to a particular theory, one aspect of the cooperative function of stacked flanges is illustrated, where the compression at the middle region 404 is sufficient to deform each inner flange 420 but the more outward flanges (i.e. the outer flange 422a and the additional flange 460 of the first plurality of flanges 416 as well as the outer flange 422b of the second plurality of flanges 418) substantially retain their shape. It is contemplated therefore that these features allow the stent 400 to better maintain engagement with the lumen wall and resist migration within the lumen.

FIG. 7 shows another embodiment of a stent 500 in which each of the pluralities of flanges 516, 518 includes an inner flange 520 adjacent to the middle region 504 and an outer flange 522 adjacent to a corresponding end. The profile of each outer flange 522 comprises an inner shoulder 534 extending from the adjacent valley 530 to a peak 536. The outer flange 522 further comprises both an outer taper 570 and a cylindrical outer section 572, in which the outer taper 570 extends toward the longitudinal axis of the hollow cylindrical body 502 to the cylindrical outer section 572, which is both concentric to said axis and parallel to the circumference 514 of the hollow cylindrical body, and which extends to the adjacent opening. In some embodiments, the outer taper 570 meets the outer section 572 at an equal or greater height above the circumference 514 as the adjacent valley.

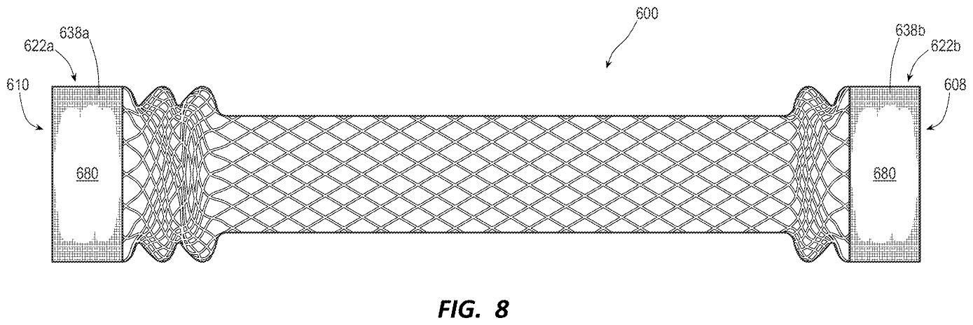

Stents as described above, may include additional features, including but not limited to anti-migration features, adhesives, and the like. For instance, a strip of a specialized material can be disposed on an external surface of the stent. In some embodiments, the specialized material provides a textured surface. In certain embodiments, the textured surface provides increased and/or decreased friction with surfaces into which the stent comes into contact, e.g., a tissue surface, as compared to the other regions of the outer surface of the stent body. In a particular example, the textured surface includes a multilayered/micropatterned surface in which friction is dependent on conditions in its environment, for example the wetness of the material. Examples of such materials are described in U.S. Patent Application Publication Nos. 2011/0311764 and 2017/0014111. In an aspect, such material can provide the stent with enhanced grip on surrounding tissues with less injury to said tissues. In some embodiments, a specialized material can be disposed on the external surface of the entirety of the stent, or of a designated portion thereof. For instance, as shown in the stent 600 in FIG. 8, the specialized material 680 may be disposed on a portion of one or both ends 608, 610 such as on one or both outer flanges 622a, 622b comprising a cylindrical outer section 638a, 638b.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “substantially” and “about.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration. All ranges also include both endpoints.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. A stent comprising:
a hollow cylindrical body having a middle region having a circumference, a diameter, and a longitudinal axis, the middle region extending to a first end defining a first opening and comprising a first plurality of flanges and to a second end defining a second opening and comprising a second plurality of flanges,
wherein each of the first plurality of flanges and the second plurality of flanges includes at least an inner flange adjacent to the middle region and an outer flange adjacent to the corresponding opening, wherein each inner flange has a profile comprising an inner shoulder extending radially from the circumference to a peak and an outer taper returning from the peak back toward the longitudinal axis to a valley.

2. The stent of claim 1, wherein each outer flange has a profile that is substantially the same as the profile of each inner flange.

3. The stent of claim 1, wherein each outer flange comprises an inner shoulder extending outward from the valley to a peak and an outer section parallel and concentric to the longitudinal axis and extending from the peak to the corresponding opening.

4. The stent of claim 1, wherein the valley defines a diameter that is greater than or equal to the diameter of the middle region.

5. The stent of claim 1, wherein at least one of the first plurality of flanges and the second plurality of flanges further includes a number of additional flanges situated between the inner flange and the outer flange, wherein said number is from 1 to 4, and wherein each of the additional flanges has a profile that is substantially the same as the profile of the inner flange.

6. The stent of claim 5, wherein each valley in the at least one of the first plurality of flanges and second plurality of flanges defines a diameter that is greater than any valleys closer to the middle region.

7. The stent of claim 5, wherein the peaks in the at least one of the first plurality of flanges and second plurality of flanges vary in height above the circumference of the middle region.

8. The stent of claim 1, wherein at least a portion of the inner shoulder of the profile of each inner flange is substantially perpendicular to the longitudinal axis of the middle region.

9. The stent of claim 1, further comprising a textured surface disposed on at least a portion of the hollow cylindrical body.

10. The stent of claim 9, wherein the textured surface is a micropatterned surface.

11. The stent of claim 9, wherein the textured surface provides increased friction with respect to a tissue surface as compared to the outer surface of the tubular body.

12. A stent comprising:
a hollow cylindrical body having a middle region having a circumference, a diameter, and a longitudinal axis, the middle region extending to a first end defining a first opening and comprising a first plurality of flanges and to a second end defining a second opening and comprising a second plurality of flanges,
wherein each of the first plurality of flanges and the second plurality of flanges includes an inner flange adjacent to the middle region and an outer flange adjacent to the corresponding opening, wherein the inner flange has a profile comprising an inner shoulder extending radially from the circumference of the middle region to a peak and an outer taper returning from the peak back toward the longitudinal axis to a valley, and
wherein the valley defines a diameter that is greater than or equal to the diameter of the middle region.

13. The stent of claim 12, wherein in at least one of the first plurality of flanges and the second plurality of flanges, the outer flange has a profile that is substantially the same as the profile of the inner flange.

14. The stent of claim 12, wherein in at least one of the first plurality of flanges and the second plurality of flanges, a profile of the outer flange comprises an inner shoulder extending outward from the circumference to a peak and an outer section parallel and concentric to the longitudinal axis and extending from the peak to the corresponding opening.

15. The stent of claim 12, wherein at least one of the first plurality of flanges and the second plurality of flanges further includes a number of additional flanges situated between the inner flange and the outer flange, wherein said number is from 1 to 4, and where each of the additional flanges has a profile that is substantially the same as the profile of the inner flange.

16. The stent of claim 12, wherein all of the peaks in the at least one of the first plurality of flanges and second plurality of flanges are substantially equal in height above the circumference of the middle region.

17. The stent of claim 12, wherein each valley in the at least one of the first plurality of flanges and second plurality of flanges defines a diameter that is greater than any valleys closer to the middle region.

18. The stent of claim 12, further comprising a textured surface disposed on at least a portion of the hollow cylindrical body.

19. The stent of claim 18, wherein the textured surface is a micropatterned surface.

20. The stent of claim 18, wherein the textured surface provides increased friction with respect to a tissue surface as compared to the outer surface of the tubular body.
